(19) United States
(12) Patent Application Publication
(10) Pub. No.: US 2012/0330342 A1
(43) Pub. Date: Dec. 27, 2012

(54) SYSTEMS AND DEVICES FOR INTRALUMENAL IMPLANTATION

(76) Inventors: Donald K. Jones, Dripping Springs, TX (US); Vladimir Mitelberg, Austin, TX (US)

(21) Appl. No.: 13/533,885

(22) Filed: Jun. 26, 2012

Related U.S. Application Data
(60) Provisional application No. 61/501,742, filed on Jun. 27, 2011.
(60) Provisional application No. 61/501,745, filed on Jun. 27, 2011.
(60) Provisional application No. 61/501,746, filed on Jun. 27, 2011.
(60) Provisional application No. 61/501,747, filed on Jun. 27, 2011.

(51) Int. Cl. A61M 29/02

(52) U.S. Cl. 606/194; 606/200

(57) ABSTRACT

Devices, systems and methods are provided for performing intra-lumenal medical procedures in a desired area of the body. Medical devices including filter devices, embolization devices, stents, delivery systems and methods of performing medical procedures to occlude, filter, redirect and or re-establish the intravascular flow of blood are provided for the treatment of hemorrhagic and ischemic disease states.

### CROSS-REFERENCE TO RELATED APPLICATIONS

This application claims the benefit of U.S. Provisional Application No. 61/501,742 filed Jun. 27, 2011, U.S. Provisional Application No. 61/501,745 filed Jun. 27, 2011 U.S. Provisional Application No. 61/501,746 filed Jun. 27, 2011 and U.S. Provisional Application No. 61/501,747 filed Jun. 27, 2011 all of which are hereby incorporated by reference herein in their entireties.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

For instance, one aspect of cerebrovascular disease in which the wall of a blood vessel becomes weakened. Under cerebral flow conditions the weakened vessel wall forms a bulge or aneurysm which can lead to symptomatic neurological deficits or ultimately a hemorrhagic stroke when ruptured. Once diagnosed a small number of these aneurysms are treatable from an endovascular approach using various embolization devices. These embolization devices include detachable balloons, coils, polymerizing liquids, gels, foams, stents and combinations thereof

The most widely used embolization devices are detachable embolization coils. These coils are generally made from biologically inert platinum alloys. To treat an aneurysm, the coils are navigated to the treatment site under fluoroscopic visualization and carefully positioned within the dome of an aneurysm using sophisticated, expensive delivery systems. Typical procedures require the positioning and deployment of multiple embolization coils which are then packed to a sufficient density as to provide a mechanical impediment to flow impingement on the fragile diseased vessel wall. Some of these bare embolization coil systems have been describe in U.S. Pat. No. 5,108,407 to Geremia, et al., entitled, “Method And Apparatus For Placement Of An Embolic Coil” and U.S. Pat. No. 5,122,136 to Guglielmi, et al., entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils at predetermined positions within vessels of the human body in order to treat aneurysms, or alternatively, to occlude the blood vessel at a particular location. Many of these systems, depending on the particular location and geometry of the aneurysm, have been used to treat aneurysms with various levels of success. One drawback associated with the use of bare embolization coils relates to the inability to adequately pack or fill the aneurysm due to the geometry of the coils which can lead to long term recanalization of the aneurysm with increased risk of rupture.

Some improvements to bare embolization coils have included the incorporation of expandable foams, bioactive materials and hydrogel technology as described in the following U.S. Pat. No. 6,723,108 to Jones, et al., entitled, “Foam Matrix Embolization Device”, U.S. Pat. No. 6,423,085 to Murayama, et al., entitled, “Biodegradable Polymer Coils for Intraluminal Implants” and U.S. Pat. No. 6,238,403 to Greene, et al., entitled, “Filamentous Embolic Device with Expansible Elements.” While some of these improved embolization coils have been moderately successful in preventing or reducing the rupture and re-rupture rate of some aneurysms, the devices have their own drawbacks. For instance, in the case of bioactive coils, the materials eliciting the biological healing response are somewhat difficult to integrate with the coil structure or have mechanical properties incompatible with those of the coil making the devices difficult to accurately position within the aneurysm. In the case of some expandable foam and hydrogel technology, the expansion of the foam or hydrogel is accomplished due to an interaction of the foam or hydrogel with the surrounding blood environment. This expansion may be immediate or time delayed but is generally, at some point, out of the control of the physician. With a time delayed response the physician may find that coils which were initially placed accurately and detached become dislodged during the expansion process leading to subsequent complications.

For many aneurysms, such as wide necked or fusiform aneurysms the geometry is not suitable for coiling alone. To somewhat expand the use of embolization coils in treating some wide necked aneurysms, stent like scaffolds have been developed to provide support for coils. These types of stent like scaffolds for use in the treatment of aneurysms have been described in U.S. Pat. No. 6,605,111 to Bose et al., entitled, “Endovascular Thin Film Devices and Methods for Treating Strokes” and U.S. Pat. No. 6,673,106 to Mitelberg, et al., entitled, “Intravascular Stent Device”. While these stent like devices have broadened the types of aneurysms amenable to embolization therapy, utilization of these devices in conjunction with embolization devices is technically more complex for the physician, may involve more risk to the patient and have a substantial cost increase for the healthcare system.

To further expand the types of aneurysm suitable for interventional radiological treatment, improved stent like devices have been disclosed in U.S. Pat. No. 5,824,053 to Khosravi et al., entitled, “Helical Mesh Endoprosthesis and Method”, U.S. Pat. No. 5,951,599 to McCrory, entitled, “Occlusion System for the Endovascular Treatment of and Aneurysm” and U.S. Pat. No. 6,063,111 to Hieshima et al., entitled, “Stent Aneurysm Treatment System and Method.” When placed across the neck of an aneurysm the proposed stent like devices purport to have a sufficient density through the wall of the device to reduce flow in the aneurysm allowing the aneurysm to clot, while at the same time having a low enough density through the wall to allow small perforator vessels adjacent to the aneurysm to remain patent. Stent devices of this nature while having the potential to reduce treatment costs have not been realized commercially due to the difficulty in manufacturing, reliability in delivering the devices to the treatment site and an inability to properly position the denser portion of the stent device accurately over the neck of the aneurysm.

Another cerebrovascular disease state is ischemia resulting from reduced or blocked arterial blood flow. The arterial blockage may be due to thrombus, plaque, foreign objects or a combination thereof Generally, plaque buildup within the lumen of the vessel, known as atherosclerotic disease, is not generally responsive to thrombolytics or mechanical disruption using guidewires. The approach to the treatment of neurovascular atherosclerotic disease has been to use modified technology developed for the treatment of cardiovascular atherosclerotic disease, such as balloons and stents, to expand the vessel at the site of the lesion to re-establish blood flow. For instance, U.S. Pat. No. 4,768,507 to Fischell et al., entitled, “Intravascular Stent and Percutaneous Insertion Catheter System for the Dilation of an Arterial Stenosis and the Prevention of Arterial Restenosis” discloses a system used for placing a coil spring stent into a vessel for the purposes of enhancing luminal dilation, preventing arterial restenosis and preventing vessel blockage resulting from intimal dissection following balloon and other methods of angioplasty. The coil spring stent is placed into spiral grooves on an insertion catheter. A back groove of the insertion catheter contains the most proximal coil of the coil spring stent which is prevented from springing radially outward by a flange. The coil spring stent is deployed when an outer cylinder is moved proximally allowing the stent to expand. Other stent systems include those disclosed in U.S. Pat. No. 4,512,338 to Balko, et al., entitled, “Process for Restoring Patency to Body Vessels”, U.S. Pat. No. 5,354,309 to Schnepp Pesch et al., entitled, “Apparatus for Widening a Body Cavity” and U.S. Pat. No. 6,833,003 to Jones et al., entitled, “Expandable Stent and Delivery System”. While the aforementioned devices may have the ability to access the cerebrovasculature, they lack sufficient structural coverage of the lesion to achieve the desired patency of the vessel without the use of a balloon device.

### SUMMARY OF THE INVENTION

In accordance with one aspect of the present invention there is provided a medical device deployment system for depositing a medical device within a body lumen of a mammal. The medical device deployment system includes a medical device, a delivery catheter and an inflation source member. The delivery catheter includes a longitudinally extending balloon member coupled to its distal end which is everted and positioned within the catheter lumen. The medical device is positioned at the distal end of the delivery catheter and disposed within the lumen of the catheter within the everted balloon member.

The balloon member of the delivery catheter is typically formed of a thin walled polymeric tube in which the distal end of the tube has been sealed and the proximal end of the balloon member is coupled to the distal end of the catheter in which the lumen of the catheter is in fluid communication with the interior surface of the balloon. The balloon member is preferably formed of a high strength non-compliant polymeric material such as nylon, polyester and others, however, metallic materials such as thin-film nitinol or other alloys may also be suitable.

The medical device takes the form of a self-expanding structure formed of a resilient material having a first constrained configuration in which the device is compressed and positioned within the lumen of the catheter and a second expanded configuration in which the device is deployed from the catheter lumen and positioned within a vessel adjacent a target site. The medical device may include proximal and distal markers to aid in positioning the device within the vasculature.

The inflation source member is coupled to the proximal end of the catheter and used to apply fluid pressure to the lumen of catheter at a level sufficient to cause the balloon member to extend longitudinally from the catheter lumen, thus deploying the stent. The preferred fluids include liquids such as saline although gases such as carbon dioxide gas may be suitable for some system configurations. The amount of fluid pressure required to inflate the balloon is in part related to the increased friction force between the balloon inner surface and the interior wall of the catheter lumen due to the outward force applied by the constrained stent device. The inflation source member preferably takes the form of a syringe (threaded or non-threaded), however other inflation sources such as a pressurized fluid sources having a valve assembly or a controllable fluid delivery pump are also suitable.

In accordance with one aspect of the present invention there is provided a medical device that takes the form of an occlusion device. The occlusion device is formed of resilient materials and preferably includes a tubular framework and an expandable polymeric material securely positioned within the tubular framework. The tubular framework may take the form of a self-expanding stent like device. The proximal and or distal ends may be flared to ensure good wall apposition and may include configurations such as barbs that aid in anchoring the device at a target site when deployed. The proximal and distal ends of the framework may include markers for visualization under fluoroscopy or MR imaging modalities. The expandable polymeric material preferably takes the form of an expandable foam plug. Alternatively the expandable material may be a swellable hydrogel that is dimensioned to occlude the lumen when deployed at a target site. As can be appreciated the swellable hydrogel may also be foamed. Suitable materials for the expandable polymeric material may include foams and or hydrogels of polyvinyl alcohol (PVA), polyacrylates, electroactive polymers, collagen, alginates, ePTFE, polymer blends or copolymers and shape memory polymers. Typically, the expandable polymeric material of this embodiment of an occlusion device is soft and compliant and generally unsuitable for remaining in position without being secured to the tubular framework. The expandable polymeric material may be secured to the framework using adhesives, thermoforming techniques or mechanical interlock. In one type of mechanical interlocking configuration, the diameter of the expandable polymeric material has a diameter equal to or greater than the diameter of the tubular framework at its distal and proximal ends, while the diameter of the mid portion of the tubular framework may be smaller than the diameter at the ends of the tubular framework. This stricture configuration ensures that the expandable polymeric material is secured within the framework unable to move distally or proximally. Another mechanical interlock configuration includes forming the expandable polymeric material within the tubular framework such the polymeric material encapsulates elements of the tubular framework thereby being secured to the framework.

In accordance with another aspect of the present invention there is provided a medical device deployment system. The medical device deployment system includes a medical device, a delivery catheter and an inflation source member. The delivery catheter includes a longitudinally extending balloon member coupled to its distal end which is everted and positioned within the catheter lumen. The medical device is positioned at the distal end of the delivery catheter and disposed within the lumen of the catheter within the everted balloon member. The delivery catheter further includes a flow restriction member positioned within the catheter lumen proximal to the everted balloon and medical device. The flow restriction member is preferably formed as tubular member having lumen substantially smaller than the lumen of the catheter. The flow restriction member may be formed of a metal, ceramic, polymer or any mixture thereof and have dimensional characteristics that do not significantly impact the ability of the catheter to access target sites within the vasculature. The flow restriction member performs the function of limiting the flow of fluid delivered from the proximal end of the catheter to the everted balloon member during deployment. This flow restriction member allows the balloon member to evert during inflation (while advancing the medical device) in a more controlled manner. As the fluid pressure applied to the lumen of the catheter reaches a sufficient level, the fluid pressure causes the balloon to begin to deploy, subsequently advancing the medical device. Once sufficient pressure is applied, the rate at which the balloon can deploy longitudinally from the catheter is dependent upon the volume of fluid available to inflate the balloon volume. The flow restriction member prevents the balloon member from inflating too rapidly (and uncontrollably deploying the medical device) by reducing the volume of fluid available for inflation thus providing more control over how the medical device is deployed.

In accordance with yet another aspect of the present invention there is provided a medical device that takes the form of a filter device. The filter device is formed of resilient materials and preferably includes a tubular framework body portion and an expandable filter assembly coupled to the body portion. The tubular framework body portion may take the form of a self-expanding stent like device. The proximal and or distal ends may be flared to ensure good wall apposition and may include configurations such as barbs that aid in anchoring the device at a target site when deployed. The proximal and distal ends of the framework body portion may include markers for visualization under fluoroscopy or MR imaging modalities. The expandable filter assembly preferably takes the form of plurality of collapsible filter arm elements coupled to the framework body portion and positioned to span the lumen diameter to capture and or break up emboli.

In accordance with still another aspect of the present invention there is provided a method of implanting a medical device in a body lumen according to an embodiment of the present invention. The method comprises the steps of: positioning a medical device deployment system within a vessel adjacent a target site; applying fluid pressure to the interior deployment lumen of the catheter; extending the balloon member from the catheter lumen longitudinally, thereby allowing a portion of the medical device to be deployed adjacent the target site; retracting the catheter relative to the deployed portion of the medical device; releasing the medical device from the delivery catheter deployment lumen; and releasing the medical device from the balloon member.

In accordance with yet still another aspect of the present invention there is provided a medical device comprising a biocompatible material. Suitable resilient materials include metal alloys such as Nitinol(NiTi), titanium, chromium alloy, stainless steel. Additional materials include polymers such as polyolefins, polyimides, polyamides, fluoropolymers, polyetheretherketone(PEEK), cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials such as polylactide polymers and polyglycolide polymers or copolymers thereof and shape memory polymers. The medical device may comprise numerous materials depending on the intended function of the device. These materials may be formed into desired shapes or attached to the device by a variety of methods which are appropriate to the materials being utilized such as laser cutting, injection molding, spray coating and casting.

In accordance with another aspect of the present invention there is provided a medical device having a coating formed of a biocompatible, bioerodible and biodegradable synthetic material. The coating may further comprise one or more pharmaceutical substances or drug compositions for delivering to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the medical device.

### BRIEF DESCRIPTION OF THE DRAWINGS

FIG. 1 is a partial cross-sectional of a medical device deployment system according to an embodiment of the present invention.

FIGS. 2A and 2B are enlarged partial cross-sectional views of the distal end of the medical device deployment system according to an embodiment of the present invention.

FIG. 3 is a side view of a deployed medical device according to an embodiment of the present invention.

FIGS. 4A through 4D are partial cross-sectional views illustrating a method of deploying a medical device within a vessel at a target site according to an embodiment of the present invention.

FIG. 5 is a side view of a deployed medical device according to another embodiment of the present invention.

FIGS. 6A through 6C are partial cross-sectional views illustrating a method of deploying another medical device within a vessel at a target site according to another embodiment of the present invention.

### DETAILED DESCRIPTION OF THE INVENTION

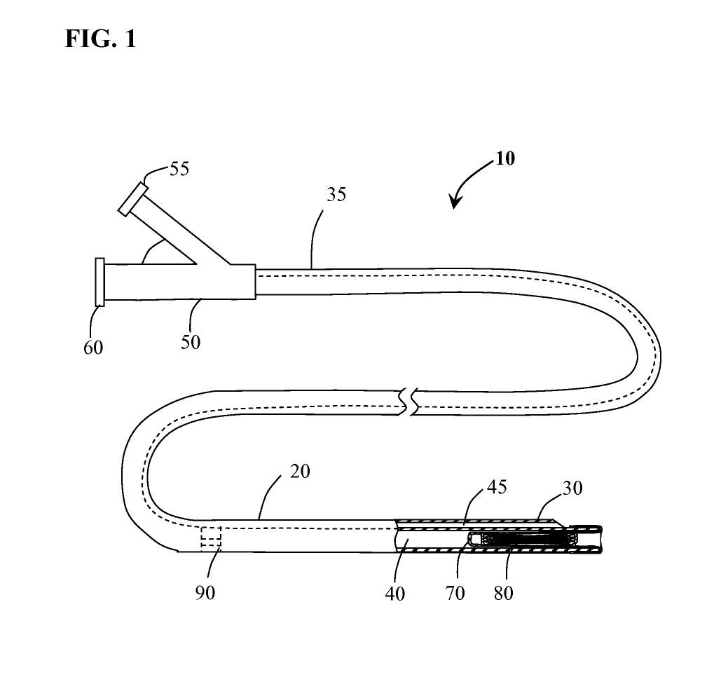

Methods and systems for implanting a medical device in a desired area of the body are herein described. FIG. 1 illustrates a medical device deployment system 10 suitable for use with embodiments of the present invention. Deployment system 10 includes an elongate tubular catheter 20 having distal and proximal ends 30 and 35 respectively, a deployment lumen 40 and a guidewire lumen 45 extending from proximal end 35 to distal end 30. A catheter hub 50 is coupled to the proximal end 35 of catheter 20. Catheter hub 50 includes a guidewire port 55 in fluid communication with guidewire lumen 45 and a deployment port 60 in fluid communication with deployment lumen 40. An elongate balloon member 70 is coupled to and positioned within deployment lumen 40 at catheter distal end 30. Occlusion device 80 is positioned within deployment lumen 40 inside of everted balloon member 70. Positioned within deployment lumen 40 of catheter 20, proximal to balloon member 70 is flow restriction member 90.

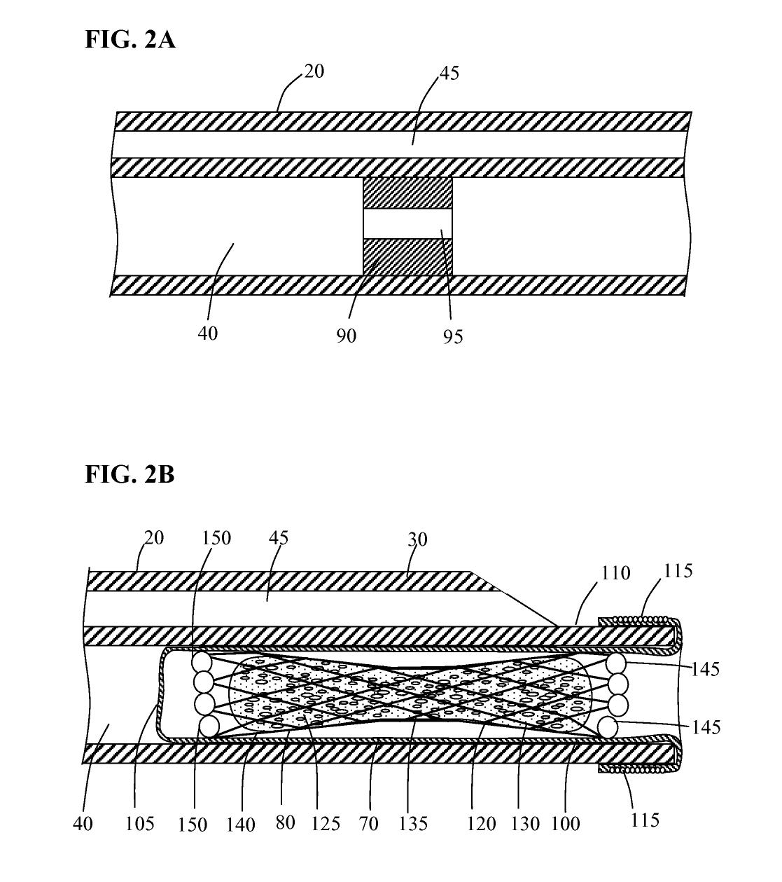

FIG. 2A depicts a magnified partial cross-sectional view of catheter 20 proximal to balloon 70. While not shown, the construction of catheter 20 may utilize known catheter technologies that incorporate braiding and or coiling using metallic or non-metallic reinforcing filamentous materials to provide high strength while maintaining catheter flexibility. The incorporation of lubricious hydrophilic and or hydrophobic materials on the inner and or outer surface of the catheter is considered to be within the scope of known catheter construction techniques and suitable for use in a deployment system of the present invention. Flow restriction member 90 having a through lumen 95 is shown secured to the inner wall of catheter 20 in deployment lumen 40. Flow restriction member 90 may be formed of any suitable material including metals, ceramics or polymers and is generally cylindrical matching the general contour of deployment lumen 40. Preferably, flow restriction member 90 is formed of a polymer such as a nylon or polyolefin and may be secured in place using known techniques such as crimping, heat fusing, ultrasonic welding or gluing. The diameter of lumen 95 is in the range of 10% to 90% of the diameter of deployment lumen 40. FIG. 2B illustrates a magnified partial cross-sectional view of distal end 30 of catheter 20. Balloon member 70 is everted and positioned within deployment lumen 40 of catheter 20 such that the inner surface of balloon member 70 is in fluid communication with deployment lumen 40 and deployment port 60 and the exterior of the balloon member is not in fluid communication with deployment lumen 40 and deployment port 60. Balloon member 70 has a proximal end 100 and a distal end 105 and is preferably formed from an elongate thin walled non-compliant material. Suitable polymeric materials include nylon or polyester tubes having a wall thickness from 0.0001 inches to 0.010 inches with a preferred range of about 0.0005 inches to 0.005 inches. Other suitable materials include metallic thin film alloys such as nitinol having a wall thickness in the range of about 0.0001 inches to about 0.001 inches. Balloon member 70 typically has a diameter less than 1.5 times the outer diameter of catheter 20 but preferably has a diameter equal to or smaller than the outer diameter of catheter 20. Balloon members of the present invention typically have a length that is substantially longer than the collapsed implantable devices, such as occlusion devices and filter devices, and range from about 40 mm to about 500 mm with a preferred range of about 50 mm to 400 mm. Optionally, balloon members of the present invention may include lubricious hydrophilic and or hydrophobic materials on the inner and or exterior surface of the balloon to reduce friction between the balloon surface and the catheter or the balloon surface and a collapsed device. The balloon proximal end 100 is coupled to catheter distal end 30 at deployment lumen tip 110 by securing member 115. Securing member 115 is shown as a flexible polymer filament wound around proximal end 100 and deployment lumen tip 110. Balloon proximal end 100 and deployment lumen tip 110 may be secured using other means such as heat fusing, ultrasonic welding and or gluing to insure a good bond and seal. The balloon distal end 105 is completed sealed using any of the aforementioned techniques and positioned within deployment lumen 40 proximal to balloon proximal end 100. Occlusion device 80 is shown in a compressed configuration within the channel defined by the exterior surface of balloon member 70 within deployment lumen 40.

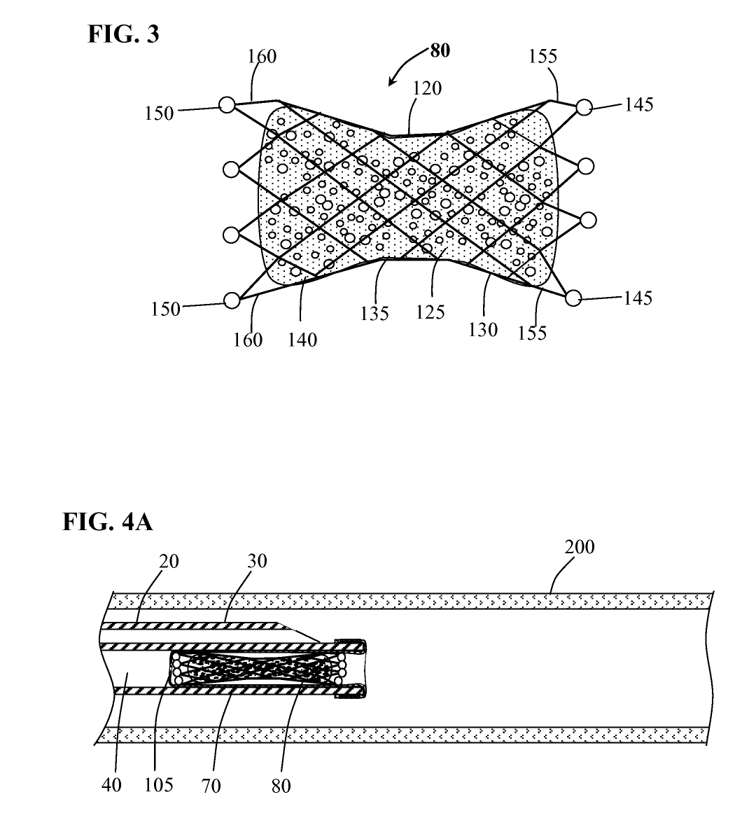

FIG. 3 illustrates a magnified view of occlusion device 80 in an expanded configuration. Occlusion device 80 has a stent like support framework 120 coupled to an expandable occlusion member 125 and has distal, intermediate and proximal portions 130,135 and 140 respectively. Occlusion device 80 also includes distal markers 145 and proximal markers 150. Preferably, the distal markers 145 are coupled to the distal flared ends 155 and the proximal markers 150 are coupled to proximal flared ends 160. While support framework 120 is shown as a simple diamond configuration, it should be noted that self expanding stent like configurations such as those described in U.S. Pat. Nos. 6,673,106 and 6,818,013 to Jones et al., entitled, “Intravascular Stent Device”, U.S. Pat. No. 5,827,321 to Roubin et al., entitled, “Non-Foreshortening Intraluminal Prosthesis”, and generally known stents having an open cell configuration are also suitable for use as a support framework to form other embodiments of an occlusion device. The size of occlusion device 80 depends upon the particular anatomy to be occluded as well as the material characteristics and design of framework 120 and occlusion member 125. Typically, the diameter of occlusion device 80 may range from about 1 mm to 50 mm and preferably between 2 mm and 15 mm.

Preferably, support framework 120 comprises a biocompatible resilient material. Suitable resilient materials include metal alloys such as nitinol, titanium, stainless steel. Additional suitable materials include polymers such as polyimides, polyamides, fluoropolymers, polyetheretherketone(PEEK) and shape memory polymers. As can be appreciated, embodiments of support framework 120 may comprise bioabsorbable and or bioerodible materials such as polycaprolactone (PCL), polyglycolic acid (PGA), polydioxanone (PDO) and combinations thereof to allow the framework to illicit a biological healing response and or to deliver pharmacological or therapeutic compounds over time. These materials may be formed into desired shapes by a variety of methods which are appropriate to the materials being utilized such as laser cutting, thermal heat treating, vacuum deposition, electro-deposition, vapor deposition, chemical etching, photo-chemical etching, electro etching, stamping, injection molding, casting, coating or any combination thereof.

Occlusion member 125 of occlusion device 80 preferably takes the form of an expandable foam plug dimensioned to occlude a desired lumen. Alternatively, occlusion member 125 may take the form of an un-foamed swellable hydrogel that is dimensioned to occlude the lumen when deployed at a target site. Suitable materials for occlusion member 125 include foams and or hydrogels of polyvinyl alcohol (PVA), polyacrylates, electroactive polymers, collagen, alginates, extracellular matrices, ePTFE, polymer blends or copolymers and shape memory polymers. Occlusion member 125 is generally soft and compliant and does not exert enough outward force to remain positioned at target site within the vasculature under flow conditions without being secured to framework 120. Occlusion member 125 may be secured to framework 120 using adhesives, thermoforming techniques or mechanical interlock configurations. FIG. 3 also illustrates a preferred type of mechanical interlocking configuration. The diameter of occlusion member 125 is equal to or greater than the diameter of the framework 120 at distal portion 130 and proximal portion 140, while the diameter of the intermediate portion 135 of framework 120 is smaller than the diameter at distal portion 130 and proximal portion 140 of framework 120. This “hour glass” configuration ensures that the occlusion member 125 is secured within the framework unable to move distally or proximally. An alternate mechanical interlock configuration includes forming occlusion member 125 within framework 120 such that the polymeric material of occlusion member 125 encapsulates strut elements of framework 120 to thereby integrally secure occlusion member 125 to the framework.

Typically, occlusion device 80 has as many markers as needed to accurately position the device depending on the particular anatomical location desired. As illustrated in FIG. 3, a plurality of markers represented by distal and proximal markers 145 and 150 are shown generally rounded as to be atraumatic to the vessel wall. Preferably, markers 145 and 150 are radio-opaque for use in fluoroscopy and formed using known materials such as gold, platinum, tantalum, tungsten, etc., however marker materials suitable for direct visual or magnetic resonance imaging are also contemplated. Markers may be attached to framework 120 using known techniques such as gluing, welding, soldering or riveting. Alternatively, markers formed of radio-opaque material may be printed, coated or electro-deposited at select locations (or the entirety) on framework 120 to provide enhanced visibility under fluoroscopy.

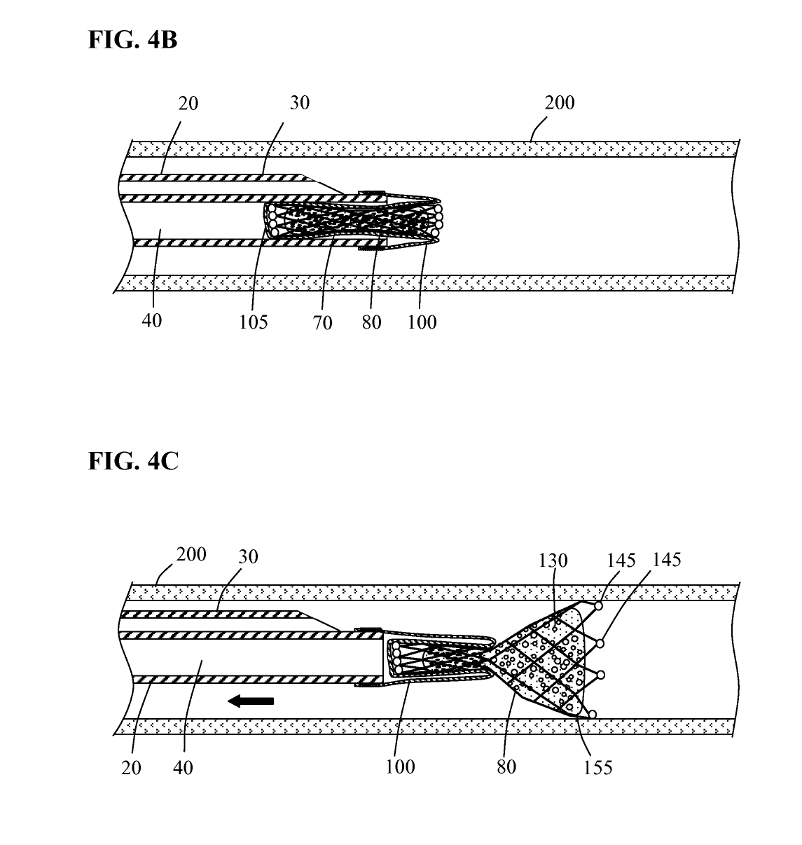

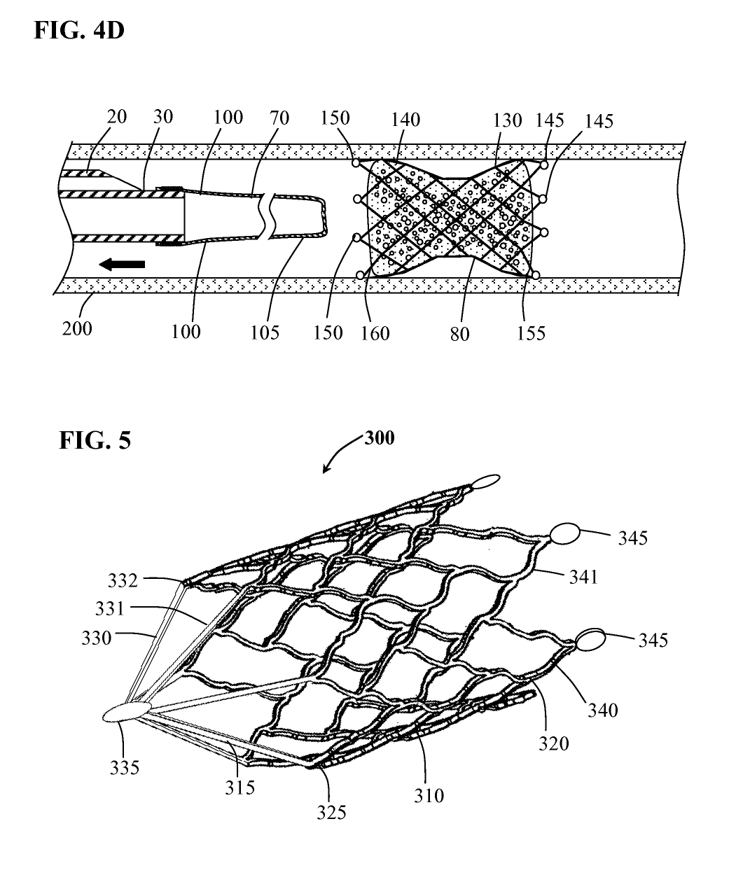

FIGS. 4A through 4D illustrate a method of deploying an occlusion device a target site within a body lumen according to one embodiment of the present invention. The deployment system 10 is positioned within a vessel 200. Catheter distal end 30 including occlusion device 80 are positioned at the target site. Occlusion device 80, being in a first constrained configuration for delivery, is positioned within everted balloon member 70 within deployment lumen 40 at distal end 30. A fluid source member such as a syringe (a fluid delivery pump or pressurized fluid source may also be suitable) is coupled to deployment port 60 of hub 50 at catheter proximal end 35. As sufficient fluid pressure is applied to deployment lumen 40, balloon member 70 begins to inflate and extend distally, thereby advancing occlusion device 80 distally. As balloon member 70 extends longitudinally, catheter 20 is retracted while a portion of occlusion device 80 exits the distal end of the catheter and the confines of balloon member 70 to move from its first constrained configuration to its second expanded configuration where distal flared ends 155 of occlusion device 80 contact the inner wall of vessel 200. As discussed previously, catheter 20 preferably includes a flow restrictor member that aids in controllably deploying occlusion device 80. Deployment of occlusion device 80 is complete after proximal flared end 160 has been deposited at the target site within the vessel. Although occlusion device 80 is in the second expanded deployment configuration occlusion device 80 has a normal unconstrained diameter which is larger than the second expanded configuration and thusly the inner diameter of vessel 200. The resilient nature of occlusion device 80, being in an expanded configuration and slightly constrained by the vessel, creates chronic outward force which is applied to the vessel wall thereby securing occlusion device 80 in position. The chronic outward of force applied by occlusion device 80 is a result of many different design attributes of the occlusion device including the dimensions and geometry of framework 120, the phase transformation temperature, Af, of the nitinol used, the shape set normal unconstrained expanded diameter of framework 120 and the expanded diameter of occlusion member 125. The balloon member 70 may be deflated and catheter 20 removed from the patient.

FIG. 5 illustrates magnified view of filter device 300 in an expanded configuration. Filter device 300 has a stent like support body 310 coupled to a filter portion 315. Support body 310 has a proximal end 320 and a distal end 325. Filter portion 315 includes a plurality of filter arms represented by filter arms 330 and 331. One end of each filter arm is coupled to the proximal end 320 of support body 310 as represented by filter arm 330 at junction 332. The other end of the filter arm is coupled to proximal tip 335 as illustrated by representative filter arms 330 and 331. Filter device 300 may also include a plurality anchor portions represented by anchor portions 340 and 341 positioned at distal end 325. Anchor portions 340 and 341 may be flared to ensure filter device 300 is secured at a target site when deployed. Alternatively, anchor portions may include barbs to aid in securing the deployed filter device. Filter device 300 also includes distal markers represented by distal marker 345. A proximal marker is preferred; however, proximal tip 335 may be formed of radio-opaque material to provide fluoroscopic visualization of the proximal end of the filter device. While support body 310 is shown as a simple diamond configuration, it should be noted that self expanding stent like configurations such as those described in U.S. Pat. Nos. 6,673,106 and 6,818,013 to Jones et al., entitled, “Intravascular Stent Device”, U.S. Pat. No. 5,827,321 to Roubin et al., entitled, “Non-Foreshortening Intraluminal Prosthesis”, and generally known stents having an open cell configuration are also suitable for use as a support body to form other embodiments of a filter device. The size of filter device 300 depends upon the particular anatomy to be filtered as well as the material characteristics and design of support body 310 and filter portion 315. Typically, the diameter of filter device 320 may range from about 1 mm to 50 mm and preferably between 2 mm and 15 mm.

Preferably, support body 310 and filter portion 315 comprise biocompatible resilient materials. Suitable resilient materials include metal alloys such as nitinol, titanium, stainless steel. Additional suitable materials include polymers such as polyimides, polyamides, fluoropolymers, polyetheretherketone(PEEK) and shape memory polymers. As can be appreciated, embodiments of support framework 120 may comprise bioabsorbable and or bioerodible materials such as polycaprolactone (PCL), polyglycolic acid (PGA), polydioxanone (PDO) and combinations thereof to allow the support body to elicit a biological healing response and or deliver pharmacological or therapeutic compounds over time. These materials may be formed into desired shapes by a variety of methods which are appropriate to the materials being utilized such as laser cutting, thermal heat treating, vacuum deposition, electro -deposition, vapor deposition, chemical etching, photo-chemical etching, electro etching, stamping, injection molding, casting, coating or any combination thereof.

A specific filter device design is heavily dependant upon the clinical application for the device and may include materials or coatings to improve the biocompatibility of the device such as coatings that include ligands adapted to capture endothelial progenitor cells within the vasculature. Additionally, the filter device may include a filter portion formed of bio-erodible or bio-absorbable materials and or materials suitable for the delivery of pharmacological or therapeutic agents adapted to reduce the formation of clots and or encourage the dissolution of thrombus or clots encountered during the intravascular flow of blood. Materials and coating process technology suitable for application to the present invention are described in U.S. Patent Application Publication No: 20070128723 A1 to Cottone et al., entitled, “Progenitor Endothelial Cell Capturing with a Drug Eluting Implantable Medical Device” herein incorporated by reference in its entirety.

Typically, filter device 300 has as many markers as needed to accurately position the device depending on the particular anatomical location desired. As illustrated in FIG. 5, a plurality of markers represented by distal markers 345 are shown generally rounded as to be atraumatic to the vessel wall. Preferably, markers 345 and proximal tip 335 are radio-opaque for use in fluoroscopy formed using known materials such as gold, platinum, tantalum, tungsten, etc., however marker materials suitable for direct visual or magnetic resonance imaging are also contemplated. Markers may be attached to body distal end 325 using known techniques such as gluing, welding, soldering or riveting. Alternatively, markers formed of radio-opaque material may be printed, coated or electro-deposited at selective locations (or its entirety) on filter device 300 to provide enhanced visibility under fluoroscopy.

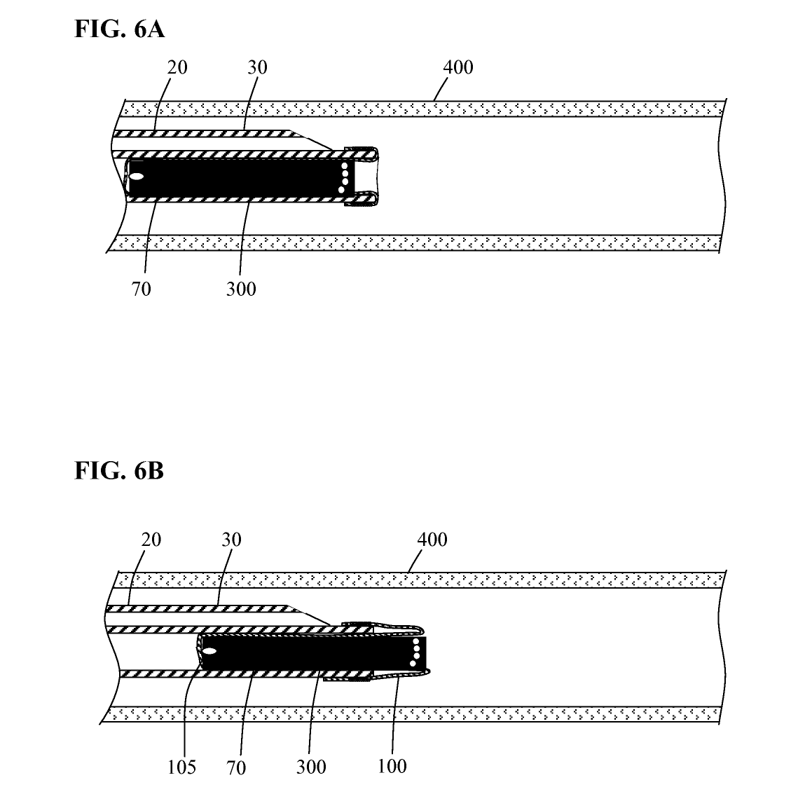

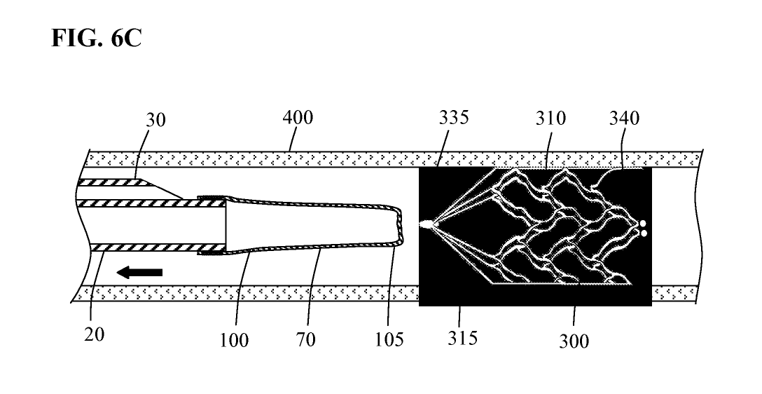

FIGS. 6A through 6C illustrate a method of deploying a filter device at a target site within a body lumen according to one embodiment of the present invention. The deployment system 10 is positioned within a vessel 400. Catheter distal end 30 including filter device 300 are positioned at the target site. Filter device 300, being in its first constrained configuration for delivery, is positioned within everted balloon member 70 within deployment lumen 40 at distal end 30. A fluid source member such as a syringe (a fluid delivery pump or pressurized fluid source may also be suitable) is coupled to deployment port 60 of hub 50 at catheter proximal end 35. As sufficient fluid pressure is applied to catheter deployment lumen 40, balloon member 70 begins to inflate and extend distally thereby advancing filter device 300 distally. As balloon member 70 extends longitudinally, catheter 20 is retracted as a portion of filter device 300 exits the distal end of the catheter and the confines of balloon member 70 to move from its first constrained configuration to its second expanded configuration where anchor portions 340 and 341 of filter device 300 contact the inner wall of vessel 400. As discussed previously, catheter 20 preferably includes a flow restrictor member that aids in controllably deploying filter device 80. Deployment of filter device 300 is complete after proximal tip 335 has been deposited at the target site within the vessel. Although filter device 300 is in the second expanded deployment configuration filter device 300 has a normal unconstrained diameter which is larger than the second expanded configuration and thusly the inner diameter of vessel 400. The resilient nature of filter device 300, being in an expanded configuration and slightly constrained by the vessel, creates chronic outward force which is applied to the vessel wall thereby securing filter device 300 in position. The chronic outward of force applied by filter device 300 is a result of many different design attributes of the filter device including the dimensions and geometry of support body 310, the phase transformation temperature, Af, of the nitinol used, and the shape set normal unconstrained expanded diameter of support body 310. The balloon member 70 may be deflated and catheter 20 removed from the patient.

Novel devices, systems and methods have been disclosed to deploy medical devices within body lumens of a mammal. Although preferred embodiments of the invention have been described, it should be understood that various modifications including the substitution of elements or components which perform substantially the same function in the same way to achieve substantially the same result may be made by those skilled in the art without departing from the scope of the claims which follow.

What is claimed is:

1. A medical device deployment system comprising:
an elongate catheter defining a longitudinal axis having proximal and distal ends and a lumen extending therethrough;
an elongate tubular balloon having an interior surface, an exterior surface, a proximal and a distal end wherein said proximal end is coupled to the distal end of said catheter and the distal end of said balloon is sealed, said balloon being everted and positioned within the lumen of said catheter whereby the distal end of said balloon is proximal to the proximal end of said balloon, said interior surface being in fluid communication with said catheter lumen and said exterior surface defining a pathway that is not in fluid communication with said catheter lumen;
a medical device formed of a resilient material and being operable between a first configuration that is compressed and constrained when positioned within said catheter lumen and a second configuration that is expanded when deployed at a target site, said medical device being positioned within said pathway in said first configuration;
an inflation source member coupled to the proximal end of said catheter for applying fluid pressure to the catheter lumen to thereby cause said everted balloon to extend distally from said catheter lumen and move said medical device from a first position within said catheter lumen to a second position distal to said catheter distal end.

2. A medical device deployment system according to claim 1 wherein said balloon comprises a metallic thin film.

3. A medical device deployment system according to claim 1 wherein said balloon has an inflated diameter less than 1.5 times the diameter of said catheter distal end.

4. A medical device deployment system according to claim 1 wherein said balloon has an inflated diameter less than the diameter of said catheter distal end.

5. A medical device deployment system according to claim 1 wherein said medical device comprises a hydrogel.

6. A medical device deployment system according to claim 1 wherein said medical device comprises a foam.

7. A medical device deployment system according to claim 1 wherein said medical device comprises nitinol.

8. A medical device deployment system according to claim 1 wherein said medical device comprises a therapeutic compound.

9. A medical device deployment system according to claim 1 wherein said medical device comprises a filter device.

10. A medical device deployment system according to claim 1 wherein said medical device comprises an occlusion device.

11. A medical device deployment system comprising:
an elongate catheter defining a longitudinal axis having proximal and distal ends and a lumen extending therethrough;
an elongate tubular balloon having an interior surface, an exterior surface, a proximal and a distal end wherein said proximal end is coupled to the distal end of said catheter and the distal end of said balloon is sealed, said balloon being everted and positioned within the lumen of said catheter whereby the distal end of said balloon is proximal to the proximal end of said balloon, said interior surface being in fluid communication with said catheter lumen and said exterior surface defining a pathway that is not in fluid communication with said catheter lumen;
a medical device formed of a resilient material and being operable between a first configuration that is compressed and constrained when positioned within said catheter lumen and a second configuration that is expanded when deployed at a target site, said medical device being positioned within said pathway in said first configuration;
an inflation source member coupled to the proximal end of said catheter for applying fluid pressure to the catheter lumen to thereby cause said everted balloon to extend distally from said catheter lumen and move said medical device from a first position within said catheter lumen to a second position distal to said catheter distal end;
a flow restrictor member having distal and proximal ends and a lumen extending therethrough, said flow restrictor member being coupled to said catheter and positioned proximal to and in fluid communication with said balloon, said flow restrictor having a lumen diameter sufficient to limit uncontrolled expansion of said balloon.

12. A medical device deployment system according to claim 11 wherein said balloon comprises a metallic thin film.

13. A medical device deployment system according to claim 11 wherein said balloon has an inflated diameter less than 1.5 times the diameter of said catheter distal end.

14. A medical device deployment system according to claim 11 wherein said balloon has an inflated diameter less than the diameter of said catheter distal end.

15. A medical device deployment system according to claim 11 wherein said medical device comprises a hydrogel.

16. A medical device deployment system according to claim 11 wherein said medical device comprises a foam.

17. A medical device deployment system according to claim 11 wherein said medical device comprises nitinol.

18. A medical device deployment system according to claim 11 wherein said medical device comprises a therapeutic compound.

19. A medical device deployment system according to claim 11 wherein said medical device comprises a filter device.

20. A medical device deployment system according to claim 11 wherein said medical device comprises an occlusion device.
